(19) United States
(12) Patent Application Publication
(10) Pub. No.: US 2026/0242408 A1
(43) Pub. Date: Aug. 20, 2026

(54) CHOLESTEROL RECOVERY FROM FISH OIL RESIDUES

(71) Applicant: AMERICAN BIOPROCESS LIMITADA, Valparaíso (CL)

(72) Inventors: Rodrigo Alejandro MANCILLA VILLALOBOS, Valparaíso (CL); Raúl Ignacio ARAVENA CONTRERAS, Providencia Santiago (CL)

(21) Appl. No.: 18/993,248

(22) PCT Filed: Jul. 19, 2022
(86) PCT No.: PCT/IB2022/056608

§ 371 Date: Jan. 10, 2025

(51) Int. Cl. C07J 9/00

(52) U.S. Cl. CPC C07J 9/00

(57) ABSTRACT

A method for separating and recovering cholesterol from fish oil residues, or saponified derivatives thereof, using a mixture of solvents comprising of water and alcohol. Additionally, a fatty acids-rich sub-fraction can be obtained which can be used for other applications such as biofuel production and animal feeding.

### OBJECT OF THE INVENTION

The invention provides a method for separating and recovering cholesterol from fish oil residues, or saponified derivatives thereof, using a mixture of solvents comprising of water and alcohol. Additionally, a fatty acids-rich sub-fraction can be obtained which can be used for other applications such as biofuel production and animal feed.

### TECHNICAL FIELD

Raw materials to be used in the present invention include saponifiable or saponified residues from fish oil refining or derivatives obtained from fish oils, such as acid oils or residues from the fractionation of fatty acids from fish oil. Particularly interesting for the invention is the use of residues derived from obtaining EPA/DHA concentrates (eicosapentaenoic acid/docosahexaenoic acid).

### BACKGROUND OF THE INVENTION

Cholesterol is a sterol synthesized by animals which performs a series of relevant functions in their physiology such as synthesis of bile acids, vitamin D, hormones, maintenance of the structure of the cell membrane, among others. In the industry, cholesterol is interesting because it is used in animal nutrition, in the production of vitamin D, and as an excipient in cosmetic and pharmaceutical products.

Currently, most of the cholesterol in the market is obtained as a by-product of the production of lanolin from sheep, and to a lesser extent from fish oil residues and by chemical synthesis.

In the prior art, there are some reported technologies for obtaining cholesterol from fish oil-derived residues. For example, the document WO 2016/096989 A1 features a method for cholesterol recovery comprising of: 1) saponification followed by: 2) extraction with at least non-water miscible solvent. The obtained product can be further purified by crystallization using at least one alcohol, an alcohol/water mixture or with an aromatic or aliphatic hydrocarbon or alcohol/hydrocarbon mixtures. The main problem related to this method is that multiple extraction steps are required to obtain high yields cholesterol recovery, making the process complex and expensive. The document U.S. Pat. No. 10,196,583 B1 discloses a process for obtaining cholesterol from fish oil comprising the steps of: a) distilling the fish oil in a vacuum column; b) distilling the first distillate in a vacuum column; c) contacting the residue of the second distillation with an alkali to saponify the same; d) extracting the saponified mixture with a non-polar organic solvent or a mixture of non-polar organic solvents; e) separating the organic phase from the aqueous phase; f) cooling the organic phase to form a solid phase and a liquid phase; and g) separating the solid phase containing the cholesterol. The main problem related to the method disclosed in U.S. Pat. No. 10,196,583 B1 is that oils contain a low amount of cholesterol, which makes the process non-economically feasible; particularly, due to the increasing demand of fish oil that has led to a constant growth in prices in recent years. Additionally, similarly to WO 2016/096989 A1, several extraction steps are required before crystallization. Furthermore, even when a distillate with a cholesterol concentration as high as 9.2% is obtained after a first distillation (example 1), a second distillation step at high vacuum and temperature conditions is required which increases the production costs and could also cause cholesterol degradation in the sample.

The document WO 2019/053744 A1 discloses a process for obtaining cholesterol from fish oil residues comprising the steps of: a) saponifying the residue in presence of 4-dimethylaminopyridine as a catalyst and then neutralizing with acid; b) heating the saponified in 2-butanone at an elevated temperature with calcium bromide to form a cholesterol adduct; c) separating the adduct followed by recrystallization in methanol. Likewise previous patent documents, several process stages and high solvent use are required. Additionally, the use of 4-dimethylaminopyridine is questionable due to its high toxicity.

The document U.S. Pat. No. 10,836,701 B2 discloses a process for obtaining cholesterol from fish oil residues comprising: a) saponifying the residue with an alkali; b) distilling the saponified mixture; c) distilling the residue of the first distillation by vacuum distillation; d) distilling the residue of the second distillation by vacuum distillation. The process leads to products with high purity and high yields. However, the use of specialized equipment (e.g., high vacuum distillation units) and high temperatures strongly increases the investment and production costs. In fact, the feeding of fatty acid salts throughout the process requires to work at temperatures above 200° C. to keep the feed as a fluid, which complicates handling of the material during the process and leads to cholesterol decomposition.

Based on the above, the present patent application sorts out the technical problem related to subjecting the feedstock to high temperatures (over 100° C.) for recovering and purifying cholesterol, which leads to lower cholesterol decomposition and lower generation of degradation products. Additionally, reduction in investment and production costs are achieved by using only one precipitation stage to obtain a solid product with a cholesterol concentration of at least of 30%, preferably at least of 50%, and more preferably at least of 70% on a dry basis. Finally, the process does not require the use of highly toxic compounds such as 4-dimethylaminopyridine.

### DESCRIPTION OF THE INVENTION

The present invention discloses a method for producing cholesterol or a cholesterol-rich fraction with a cholesterol concentration of at least of 30%, preferably at least of 50%, and more preferably at least of 70% on a dry basis from fish oil residues containing at least 5% free or esterified cholesterol, and at least 50% free or esterified fatty acids.

The present invention preferably uses, but is not limited to, residues from the production of EPA+DHA concentrates from fish oils due to the high cholesterol concentration in theses residues. Preferably, but not limited to them, vacuum distillation bottoms may be used, which may exhibit cholesterol concentrations from 5 to 50% depending on the process from which they are obtained as shown in U.S. Pat. No. 10,196,583 B1, U.S. Pat. No. 10,836,701 B2 and WO 2019/053744 A1.

According to the invention, the method has the advantage of avoiding state-of-the-art processes that involve high thermal stress and lead to the decomposition of cholesterol, reducing generation of undesirable by-products and increasing cholesterol recovery. Additionally, the proposed method requires fewer unit operations than other processes described in the state of the art that resort to a series of distillations and crystallizations. Fewer unit operations lead to lower investment and production costs.

The present invention provides a method of separating and recovering cholesterol from fish oil residues, or saponified derivatives thereof, using a mixture of solvents comprising of water and alcohol. A fatty acids rich sub-fraction is obtained from the cholesterol recovery process. This sub-fraction can be used for other applications such as biofuel production and animal feeding.

The method of the present invention comprises the stages of:
a) saponifying a fish oil residue using an alkali in water or an alkali in a solution of water and alcohol;
b) adding a solvent selected from the group formed by water or alcohol or a solution of water and alcohol to the mixture obtained in stage a) up to a ratio of solvent to residue from 15 to 30 w/w;
c) maintaining the mixture obtained in stage b) at a temperature lower than the temperature of stage b) to precipitate cholesterol-rich solid;
d) recovering the cholesterol-rich solid from the mixture after stage c); and
e) washing the cholesterol-rich solid with a washing solution comprising water and alcohol.

Optionally, part of the used alkali may be neutralized using an acid after the saponification reaction in stage a) and before the stage c).

Optionally, stage b) may be carried out in a series of stages until a solvent to residue ratio of 15 to 30 w/w is reached.

In another object of the present invention, it is provided a method in which stages a) and b) may be changed by the following sequence of operations:
a.1) saponify a fish oil residue using an alkali in water or an alkali in a solution of water and alcohol;
a.2) add an acid to the solution obtained in a.1) up to a pH between 2 and 6;
a.3) recover an oily phase after adding the acid in a.2), and removing the aqueous phase and the precipitated salts obtained;
a.4) mix the oily phase obtained in a.3) with a solvent comprising an alkali and a solution of water and alcohol up to a ratio of solvent to residue from 15 to 30 w/w;
a.5) maintain the mixture obtained in stage a.4) to a temperature lower than the temperature of stage a.4) to precipitate cholesterol-rich solid;
a.6) recover the cholesterol-rich solid from the mixture after stage a.5); and
a.7) wash the cholesterol-rich solid with a washing solution comprising water or a water and alcohol solution.

In the context of the present invention, without limiting the scope thereof, the term “solution of water and alcohol” means a mixture comprising water and at least one alcohol.

By applying the proposed method, it is possible to obtain a cholesterol-rich solid in stages e) and a.7) with a cholesterol concentration of at least of 30%, preferably at least of 50%, and more preferably at least of 70% on a dry basis with only one precipitation stage.

The method for obtaining the cholesterol concentrate leads to produce a liquid stream comprised of the solvent and the residue which has not precipitated in stage c) or a.5). Additionally, another liquid stream or spent washing solution is generated in stage e) or a.7), which is composed by the washing solution and part of the residue which has not precipitated. Both liquid streams could be evaporated or distilled to recover the solvent, but the high volume of solvent used would lead to high operation costs related to the energy consumption. Consequently, this invention proposes a solvent recovery process after washing (stages e) and a.7)) based on the following stages:
f) add an acid to the liquid stream obtained in step d) or a.6) after removing the cholesterol-rich solid and to the spent washing solution of step e) or a.7) up to a pH between 2 and 6 is obtained; and
g) recover an oily phase obtained after adding the acid in step f) and remove a non-oily liquid phase and salts obtained.

Optionally, the non-oily liquid phase may be maintained to a low temperature to precipitate a solid residue and then the solid residue formed therein may be removed. These stages produce a non-oily liquid phase rich in water and alcohol that can be reused in the process in any of step a) and/or b) and/or e) and/or a.1) and/or a.4) and/or a.7) without using evaporation and/or distillation after each operation for recovering the water and alcohol. On the other hand, the oily phase obtained in this process is rich in fatty acids, so this oily phase can be used in other applications such as biofuel and animal feeding.

For the saponification of the residue in the stage a) or a. 1), any known method in the prior art can be used, without limiting the scope of the present invention. Preferably, for example, saponification using a solution of water and alcohol with sodium hydroxide and/or potassium hydroxide as alkali are recommended due to their high efficiencies and low cost.

Preferably, the saponifying a fish oil residue using an alkali in water or an alkali in a solution of water and alcohol is carried out at a temperature of at least over 65° C. Preferably, the method is characterized in that the mixtures or dispersions obtained in stage b) and/or in stage a.4) have a pH over 9.

Preferably, the method is characterized in that after the stages b) or a.4), the mixture has an alcohol/water ratio at least between 0.7 and 5 w/w. Preferably, if ethanol is the only alcohol used, the ethanol/water ratio is at least between 0.8 and 4 w/w.

Preferably, the method comprises maintaining the mixture obtained in steps c) or a.5) at a temperature that is lower than 30° C. to precipitate solid rich in cholesterol.

Preferably, the mixture obtained in steps c) or a.5) is maintained at a temperature that is lower than 30° C. for at least 15 minutes, preferably at least 30 minutes and more preferably at least 60 minutes.

Preferably, the method is characterized in that the alcohol to be used in the water/alcohol solution may be, but is not limited to, methanol, ethanol, n-propanol, isopropanol, n-butanol, sec-butanol, tert-butanol, iso-butanol, ethylene glycol, diethylene glycol, diacetone alcohol or a mixture thereof. Methanol and ethanol are preferred due to their lower boiling point which makes them easier to be removed from the cholesterol-rich solid. Preferably, ethanol is used when by-products from ethyl-ester production from fish oil are used as raw material.

Preferably, the method is characterized in that in acidulation or neutralization of the alkali, any organic or inorganic acid such as: hydrochloric acid, sulfuric acid, boric acid, citric acid, lactic acid, phosphoric acid, nitric acid, acetic acid, propionic acid, among others, may be used.

The cholesterol-rich solid can be recovered in stage d) or a.6) by using any known method in the prior art for separation of solids from liquid streams such as filtration and centrifugation, without limiting the scope of the application.

Preferably, the method is characterized in that the precipitation and separation in stages c) and d) or a.5) and a.6) can be carried out in 2 or more successive steps at different temperatures wherein these temperatures are lower than 30° C.

By applying the proposed method, it is possible to obtain a cholesterol-rich solid in stage e) or a.7) with a cholesterol concentration above 30% on a dry basis. More preferably the cholesterol concentration in said solid is at least of 50%, and more preferably at least of 70% on a dry basis with only one precipitation.

Advantageously, the method is characterized in that the cholesterol recovery efficiency in stage e) or a.7) is at least 25%, preferably at least 50% of the cholesterol in the fish oil residue.

The cholesterol-rich solid obtained in stage e) can be dried to obtain a dry or low-moisture solid using any known drying technique in the state of the art, such as spray drying, rotary drum, trays, tape, among others.

The cholesterol-rich solid of stage e) can optionally be purified by crystallization to increase the cholesterol concentration. Preferably, a mixture of water, alcohol, and hydrocarbon is used in the crystallization.

### EXAMPLES OF APPLICATION

### Example 1. Recovery of Cholesterol from Residue with 11.1% Cholesterol

50 g of fish oil residue from the EPA and DHA concentrate production process were saponified using 15.3 g of NaOH in a solution with 26.3 g of water and 101 g of ethanol at 85° C. for 5 hours. Then, the solution was neutralized using a 50% H₂SO₄ ethanolic solution up to a pH between 3.3-3.5. The formed oily phase was recovered and dried in a rotary evaporator (100-200 mbar) at 85° C. The oily phase contained 11.1% cholesterol (analyzed using gas chromatography) and an acid value of 102 mg NaOH/g.

A series of precipitation experiments using ethanol or methanol were carried out and the results are summarized in Table 1. To carry out the experiments, 4 g of sample were mixed with the amounts of ethanol or methanol, water, and 514.8 mg NaOH shown in the table at 60° C. for 1 hour. Then, the mixture was cooled down up to the precipitation temperature (Tp) and maintained at that temperature for 3 hours. After 3 hours, the sample was centrifuged at 1,000 rpm for 10 minutes and the supernatant was removed. The solid was washed with 4 g of a solution of water and alcohol with a composition identical to the solution used in the precipitation. Finally, the solid was recovered by dissolving it in hexane, and the solution was transferred to a round bottom flask. Hexane was removed using a rotary evaporator (100-200 mbar) at 85° C. The moisture content of the solid was measured by drying the solid at 105° C. in an oven up to constant weight, and the cholesterol content was measured by gas chromatography.

TABLE 1
Precipitation conditions and results
obtained for sample of Example 1

| | Ratio | Ratio | Tp | Recovery | Purity |
|---|---|---|---|---|---|
| Alcohol | Solvent/sample | Alcohol/water | (° C.) | (%) | (%) |
| E | 15 | 0.43 | 5 | 30.4 | 48.2 |
| E | 19 | 0.43 | 5 | 30.5 | 39.2 |
| E | 19 | 1.24 | 5 | 56.0 | 66.4 |
| E | 19 | 0.85 | 5 | 50.1 | 62.8 |
| M | 15 | 0.68 | 5 | 44.5 | 38.9 |
| M | 19 | 1.24 | 5 | 50.1 | 50.9 |
| M | 15 | 1 | 10 | 65.5 | 51.5 |
| M | 15 | 1.26 | 10 | 73.0 | 56.5 |

Tp: precipitation temperature.
Alcohol: E (ethanol) or M (methanol).
Recovery: recovery of cholesterol in relation to those present in the raw material

The results in Table 1 show that different alcohols can be used to carry out cholesterol recovery from fish oil residues. According to these results, it is evident that from the operating conditions disclosed in the present application it is possible to achieve a good recovery and purity of the cholesterol obtained from fish oil. Additionally, our results show that NaOH can be used as alkali.

### Example 2. Recovery of Cholesterol from Residue with 12.5% Cholesterol

A fish oil residue from the EPA/DHA concentrate production process was treated using the same procedure used in Example 1. After the procedure, the oily phase contained 12.5% cholesterol and an acid value of 113 mg NaOH/g. To carry out the experiments, 4 g of sample were mixed with the amounts of alcohol, water and 700 mg KOH shown in Table 2 at 60° C. for 1 hour. Other precipitation conditions were evaluated, and the results of the precipitation are shown in Table 2. The precipitation procedure was the same followed in Example 1. The solid was washed with 16 g of a solution of water and alcohol with a composition identical to the solution used in the precipitation.

TABLE 2
Precipitation conditions and results
obtained for sample of Example 2.

| | Ratio | Ratio | Tp | Recovery | Purity |
|---|---|---|---|---|---|
| Alcohol | Solvent/sample | Alcohol/water | (° C.) | (%) | (%) |
| E | 15 | 0.68 | 5 | 27.7 | 66.4 |
| E | 15 | 0.52 | 5 | 3.6 | 67.1 |
| E | 19 | 0.85 | 5 | 57.3 | 73.5 |
| E | 19 | 1.5 | 5 | 67.4 | 82.8 |
| E | 25.1 | 1.5 | 5 | 63.9 | 75.2 |
| M | 15 | 1.25 | 5 | 62.5 | 76.7 |
| M | 15 | 1.5 | 5 | 67.1 | 76.5 |

M: methanol; E: ethanol.
Tp: precipitation temperature.
Alcohol: E (ethanol) or M (methanol).
Recovery: recovery of phytosterols in relation to those present in the raw material

The results in Table 2 show that different alcohols can be used to carry out cholesterol recovery from fish oil residues. According to these results, it is evident that from the operating conditions disclosed in the present application it is possible to achieve a good recovery and purity of the cholesterol obtained from fish oil. Our results also show that KOH can be used as alkali.

### Example 3. Recovery of Cholesterol from Residue with 28.7% Cholesterol

A fish oil residue from the EPA/DHA concentrate production process was treated using the same procedure used in Example 1. In this case, the oily phase contained 28.7% cholesterol and an acid value of 85.3 mg NaOH/g. To carry out the experiments, 3 g of sample were mixed with the amounts of alcohol, water, and 418 mg NaOH shown in Table 3 at 60° C. for 1 hour. The precipitation procedure was the same followed in Example 1. The results obtained from the precipitation are shown in Table 3.

TABLE 3
Precipitation conditions and results
obtained for sample of Example 3.

| | Ratio | Ratio | Tp | Recovery | Purity |
|---|---|---|---|---|---|
| Alcohol | Solvent/sample | Alcohol/water | (° C.) | (%) | (%) |
| E | 19 | 1.25 | 5 | 59.5 | 82.1 |
| E | 19 | 0.85 | 5 | 52.1 | 80.6 |
| E | 15 | 0.43 | 5 | 48.7 | 51.4 |
| M | 15 | 0.68 | 5 | 41.6 | 35.2 |
| M | 17.5 | 1.22 | 5 | 62.0 | 61.4 |

The results in Table 3 show that methanol and ethanol can be used to carry out cholesterol recovery from fish oil residues. According to these results, it is evident that from the operating conditions disclosed in the present application it is possible to achieve a good recovery and purity of the cholesterol obtained from fish oil.

### Example 4. Recovery of Cholesterol from Samples Directly after Saponification

50.0 g of fish oil residue from the EPA and DHA concentrate production process (Example 3) were mixed with 15.2 g of NaOH and 125 g of a solution with 58.8% of ethanol in water. In this case ethanol was preferred due to ethanol is formed after fatty acid ethyl ester hydrolysis. The mixture was refluxed and stirred at 1,000 rpm for 4 hours to saponify the sample. The mixture was then cooled down. Ethanol and water were added to reach a solvent/sample ratio of 20 and an ethanol/water ratio of 1.42. Then the mixture was cooled down up to 15° C. and maintain for 60 minutes. The solid formed was centrifugated and then washed with 200 g of solution with 58.8% of ethanol in water. Solid rich in cholesterol was recovered with 82.5% cholesterol in dry based (cholesterol recovery=88.1%).

50.1 g of fish oil residue from the EPA and DHA concentrate production process (Example 3) were saponified using NaOH and 125 g of a solution with 79% ethanol in water. The mixture was then cooled down. Ethanol and water were added to reach a solvent/sample ratio of 17.5 and an ethanol/water ratio of 3.83. Then the mixture was cooled down up to 15° C. and maintain for 60 minutes. The solid formed was centrifugated and then washed with 200 g of solution with 79% of ethanol in water. Solid rich in cholesterol was recovered with 90.0% cholesterol in dry based (cholesterol recovery=84.6%).

50.0 g of fish oil residue from the EPA and DHA concentrate production process (Example 1) were mixed with 15.0 g of NaOH and 125 g of a solution with 59.7% of ethanol in water. The mixture was refluxed and stirred at 1,000 rpm for 4 hours to saponify the sample. The mixture was then cooled down. Ethanol and water were added to reach a solvent/sample ratio of 19 and an ethanol/water ratio of 1.5. Then the mixture was cooled down up to 5° C. and maintain for 60 minutes. The solid formed was centrifugated and then washed with 100 g of solution with 59.7% of ethanol in water. Solid rich in cholesterol was recovered with 64.2% cholesterol in dry based (cholesterol recovery=71.7%).

50.0 g of fish oil residue from the EPA and DHA concentrate production process (Example 2) were mixed with 15.0 g of NaOH and 125 g of a solution with 59.7% of ethanol in water. The mixture was refluxed and stirred at 1,000 rpm for 4 hours to saponify the sample. The mixture was then cooled down. Ethanol and water were added to reach a solvent/sample ratio of 19 and an ethanol/water ratio of 1.5. Then the mixture was cooled down up to 5° C. and maintain for 60 minutes. The solid formed was centrifugated and then washed with 200 g of solution with 59.7% of ethanol in water. Solid rich in cholesterol was recovered with 77.2% cholesterol in dry based (cholesterol recovery=86.4%).

### Example 5. Crystallization of Cholesterol-Rich Solids

0.5 g of cholesterol-rich solid obtained as described by this patent application and with a purity of 84.8% cholesterol were crystalized. The sample was dissolved in a mixture with 2.5 g of heptane, 0.25 g of methanol and 0.5 g of water at 60° C. Then, the mixture was cooled down at 15° C. and maintained at 15° C. for 2 hours. Finally, the crystallized solids were filtered and washed with 5 g of methanol. A final product with 92.8% of cholesterol (cholesterol recovery=69.2%). A similar experiment was performed using the same cholesterol-rich solid, but 5 g of heptane were used instead of methanol in the washing step. A product with 91.7% of cholesterol was recovered (cholesterol recovery=85.2%).

Similarly, 0.5 g of a cholesterol-rich solid with 78.9% of cholesterol was crystallized using 2.5 g of heptane, 0.25 g of methanol and 0.5 g of water at 15° C. for 2 hours. A product with 93.2% of cholesterol was recovered (cholesterol recovery=75.6%).

1. A method for separating and recovering cholesterol from fish oil residues, or saponified derivatives thereof, comprising the stages of:
a) saponifying a fish oil residue using an alkali in water or an alkali in a solution of water and alcohol;
b) adding a solvent selected from the group formed by water or alcohol or a solution of water and alcohol to the mixture obtained in stage a) up to a ratio of solvent to residue from 15 to 30 w/w;
c) maintaining the mixture obtained in stage b) at a temperature lower than the temperature of stage b) to precipitate cholesterol-rich solid;
d) recovering the cholesterol-rich solid from the mixture after stage c); and
e) washing the cholesterol-rich solid with a washing solution comprising water and alcohol.

2. The method of separating and recovering cholesterol according to claim 1, wherein the saponification is carried out using sodium hydroxide, potassium hydroxide or a mixture of thereof as alkali.

3. The method of separating and recovering cholesterol according to claim 1, wherein the solution of water and alcohol of stage b) has an alcohol/water ratio at least between 0.7 to 5 w/w.

4. The method of separating and recovering cholesterol according to claim 3, wherein the alcohol is ethanol and the ethanol/water ratio is at least between 0.8 and 4 w/w.

5. The method of separating and recovering cholesterol according to claim 1, wherein part of the alkali may be partially neutralized using an acid after the saponification reaction in stage a) and before the stage c).

6. The method of separating and recovering cholesterol according to claim 5, wherein the acid is organic or inorganic acid selected from the group consisting of: hydrochloric acid, sulfuric acid, boric acid, citric acid, lactic acid, phosphoric acid, nitric acid, acetic acid, and propionic acid.

7. The method of separating and recovering cholesterol according to claim 1, wherein the alcohol is selected from the group consisting of: methanol, ethanol, n-propanol, isopropanol, n-butanol, sec-butanol, tert-butanol, iso-butanol, ethylene glycol, diethylene glycol, diacetone alcohol and a mixture thereof.

8. The method of separating and recovering cholesterol according to claim 1, wherein step b) is carried out in at least 2 stages to obtain a solvent to residue ratio from 15 to 30 w/w.

9. The method of separating and recovering cholesterol according to claim 1, wherein the solution or dispersion of stage b) has a pH above 9.

10. The method of separating and recovering cholesterol according to claim 1, wherein stages c) and d) are carried out in 2 or more successive steps at different temperatures.

11. The method of separating and recovering cholesterol according to claim 1, wherein the recovery of the cholesterol-rich solid is carried out by a method for separation of solids from liquid streams, wherein the method for separation of solids from liquid streams is filtration or centrifugation.

12. The method of separating and recovering cholesterol according to claim 1, wherein the solvent of the liquid stream of stage c) and the washing solution of stage e) are recovered by the following steps:
f) adding an acid to the liquid stream obtained in d) after removing the cholesterol-rich solid and to the spent washing solution in e) up to a pH between 2 and 6 is obtained; and
g) recovering an oily phase obtained after adding the acid in step f) and removing a non-oily liquid phase and salts obtained therefrom.

13. The method of separating and recovering cholesterol according to claim 12, wherein the non-oily liquid phase is maintained to a low temperature to precipitate a solid residue and the solid residue formed is removed.

14. The method of separating and recovering cholesterol according to claim 12, wherein the oily phase obtained is rich in fatty acids and is used as biofuel or in animal feed.

15. A method for separating and recovering cholesterol from fish oil residues, or saponified derivatives thereof, comprising the stages of:
a.1) saponifying a fish oil residue using an alkali in water or an alkali in a solution of water and alcohol;
a.2) adding an acid to the solution obtained in a.1) up to a pH between 2 and 6;
a.3) recovering an oily phase after adding the acid in a.2), and removing the aqueous phase and the precipitated salts obtained;
a.4) mixing the oily phase obtained in a.3) with a solvent comprising an alkali and a solution of water and alcohol up to a ratio of solvent to residue from 15 to 30 w/w;
a.5) maintaining the mixture obtained in stage a.4) to a temperature lower than the temperature of stage a.4) to precipitate cholesterol-rich solid;
a.6) recovering the cholesterol-rich solid from the mixture after stage a.5); and
a.7) washing the cholesterol-rich solid with a washing solution comprising water or a water and alcohol solution.

16. The method of separating and recovering cholesterol according to claim 15, wherein the saponification is carried out using sodium hydroxide, potassium hydroxide or a mixture thereof as alkali.

17. The method of separating and recovering cholesterol according to claim 15, wherein the solution of water and alcohol of stage a.4) has an alcohol/water ratio at least between 0.7 to 5 w/w.

18. The method of separating and recovering cholesterol according to claim 17, wherein the alcohol is ethanol and in that the ethanol/water ratio is at least between 0.8 and 4 w/w.

19. The method of separating and recovering cholesterol according to claim 15, wherein the oily phase obtained is rich in fatty acids and is used as biofuel or animal feed.

20. The method of separating and recovering cholesterol according to claim 15, wherein the acid organic or inorganic acid selected from the group consisting of hydrochloric acid, sulfuric acid, boric acid, citric acid, lactic acid, phosphoric acid, nitric acid, acetic acid, and propionic acid.

21. The method of separating and recovering cholesterol according to claim 15, wherein the alcohol is selected from the group consisting of methanol, ethanol, n-propanol, isopropanol, n-butanol, sec-butanol, tert-butanol, iso-butanol, ethylene glycol, diethylene glycol, diacetone alcohol and a mixture thereof.

22. The method of separating and recovering cholesterol according to claim 15, wherein the recovery of the cholesterol-rich solid is carried out by method for the separation of solids from liquid streams, wherein the method for such as filtration and centrifugation.

23. The method of separating and recovering cholesterol according to claim 15, wherein the solvent of the liquid stream of stage a.5) and the washing solution of stage a.7) are recovered by the following steps:
f) add an acid to the liquid stream obtained in a.6) after removing the cholesterol-rich solid and to the spent washing solution in a.7) up to a pH between 2 and 6 is obtained;
g) recover an oily phase obtained after adding the acid in step f) and removing a non-oily liquid phase and salts obtained therefrom.

24. The method of separating and recovering cholesterol according to claim 23, wherein the non-oily liquid phase is maintained to a low temperature to precipitate a solid residue and the solid residue formed is removed.

25. The method of separating and recovering cholesterol according to claim 23, wherein the oily phase obtained is rich in fatty acids and is used in as biofuel or in animal feed.

26. The method of separating and recovering cholesterol according to claim 15, wherein stages a.5) and a.6) can be carried out in 2 or more successive steps at different temperatures.

27. The method of separating and recovering cholesterol according to the claim 26, wherein the cholesterol-rich solid is purified by crystallization using a mixture of water, alcohol, and hydrocarbon.
